(19) United States
(12) Patent Application Publication
(10) Pub. No.: US 2018/0271380 A1
(43) Pub. Date: Sep. 27, 2018

(54) RESPIRATION RATE MONITORING BY MULTIPARAMETER ALGORITHM IN A DEVICE INCLUDING INTEGRATED BELT SENSOR

(71) Applicant: KONINKLIJKE PHILIPS N.V., EINDHOVEN (NL)

(72) Inventors: Richard E. Gregg, Westford, MA (US); Juan Brea, Andover, MA (US)

(21) Appl. No.: 15/535,563

(22) PCT Filed: Dec. 7, 2015
(86) PCT No.: PCT/IB2015/059394

§ 371 Date: Jun. 13, 2017

(51) Int. Cl. A61B 5/0205; A61B 5/0428; A61B 5/00

(52) U.S. Cl. CPC A61B 5/0205; A61B 5/04286; A61B 5/6823; A61B 5/0245; A61B 5/7278; A61B 5/742; A61B 5/6831

(57) ABSTRACT

A physical monitoring system for monitoring and measuring a patient's respiration is described. The system includes one or more resistive or inductive respiration belts (110a, 10b), an electronic monitoring device (102) with a processor programmed to compute respiration and a module retainer (104) for accommodating the electronic monitoring device and securing the electronic monitoring device to the one or more resistive or inductive respiration belts. The system further includes electrocardiogram (ECG) electrodes (108) attached to or embedded in theone or more resistive or inductive respiration belts. The ECG electrodes are connected with the electronic monitoring module (102) via wires passing through the belts. The system can further include an accelerometer (204) integrated with one or more of the ECG electrodes that are attached to or embedded in theone or more resistive or inductive respiration belts.

### FIELD

The following relates generally to the medical monitoring arts. It finds particular application with a device for monitoring and calculating respiration in a user and will be described with particular reference thereto. However, the present disclosure will find applications in other areas as well.

### BACKGROUND

Accurate and reliable patient monitoring in hospitals is essential to providing necessary care to patients in medical facilities. Hospitals, nursing homes, and other medical facilities typically use systems that measure respiration rate using a single sensor or algorithm or use more inaccurate methods such as manual counting of a patient's breath. It is important to calculate up to date respiration for a patient as respiration rate may be an early sign of a decline in a patient's health. Respiration rate can be measured manually (i.e. counting visually observed breaths) or using automated devices such as belts to measure chest expansion. However, these approaches tend to be inaccurate at low respiratory rate, are bulky and inconvenient to use, and may be affected by patient motion.

Another known approach is the use of an accelerometer to measure chest motion, which advantageously has a smaller form factor than a respiratory belt. However, an accelerometer-based respiratory rate monitor can also be affected by patient motion, as well as by the precise placement of the accelerometer on the chest.

These respiratory rate monitors can also interfere with other patient monitor devices that are commonly used along with a respiratory monitor, such as electrocardiograph (ECG). Wiring for these various devices can become tangled, and generally inconveniences the patient. This has led to increased use of wireless patient monitors, but these have issues of their own, such as the possibility of cross-talk between monitoring devices, and possible wireless signal interference. The lack of physical wired connections can also make it difficult to verify that the wireless patient monitor is properly connected.

### SUMMARY

The present disclosure overcomes the above mentioned shortcomings of current respiration measurement and monitoring systems.

In accordance with one aspect, a physical monitoring system is described. The system includes one or more resistive or inductive respiration belts configured to be disposed around the chest to detect chest expansion and contraction during breathing. An electronic monitoring module is operatively connected with the one or more resistive or inductive respiration belts and comprises a processor programmed to compute respiration using the one or more resistive or inductive respiration belts. A module retainer receives the electronic monitoring module and secures the electronic monitoring module to the one or more resistive or inductive respiration belts.

In accordance with another aspect, a physical monitoring system is described, comprising: one or more resistive or inductive respiration belts; electrocardiogram (ECG) electrodes attached to or embedded in the one or more resistive or inductive respiration belts; an electronic monitoring module attached to the one or more resistive or inductive respiration belts and to the ECG electrodes via wires passing through the one or more resistive or inductive respiration belts, the electronic monitoring module programmed to compute respiration using at least the one or more resistive or inductive respiration belts and to compute at least heart rate using the ECG electrodes; and a module retainer configured to receive the electronic monitoring module and to secure the electronic monitoring module to the one or more resistive or inductive respiration belts.

In accordance with another aspect, a physical monitoring system is described, comprising: a wearable frame including one or more resistive or inductive respiration belts supported by shoulder straps; electrocardiogram (ECG) electrodes attached to or embedded in the wearable frame; an electronic monitoring module configured to measure respiration rate and heart rate using sensors including at least the one or more resistive or inductive respiration belts and the ECG electrodes; and a module retainer configured to receive the electronic monitoring module and to secure the electronic monitoring module to the wearable frame.

One advantage resides in improved monitoring and calculation of a patient's respiration rate based upon additional incorporated patient data.

Another advantage resides in improved and less expensive monitoring devices.

Another advantage resides in reduced patient inconvenience when being monitored by multiple monitoring devices.

Still further advantages of the present invention will be appreciated to those of ordinary skill in the art upon reading and understand the following detailed description. It is to be appreciated that none, one, two, or more of these advantages may be achieved by a particular embodiment.

### BRIEF DESCRIPTION OF THE DRAWINGS

The invention may take form in various components and arrangements of components, and in various steps and arrangement of steps. The drawings are only for purposes of illustrating the preferred embodiments and are not to be construed as limiting the invention.

FIG. 1 illustrates an embodiment of the physical monitoring device system.

FIG. 2 illustrates an embodiment of the accelerometers and ECG electrodes used for detecting respiration in a patient.

FIG. 3 illustrates a block diagram indicating the various inputs and outputs for calculating a patient's respiration rate.

FIG. 4 is illustrates an embodiment of the electronic monitoring device.

### DETAILED DESCRIPTION

Disclosed herein are improved patient monitoring systems for more accurate calculation and monitoring of a patient's respiration rate while in a medical facility.

The present systems can be used in a variety of institutions such as hospitals, hospital and patient care systems, clinics, nursing homes, and the like. Accordingly, “hospital” is used in the following for simplicity of discussion, “hospital” is to be understood as including all such medical institutions.

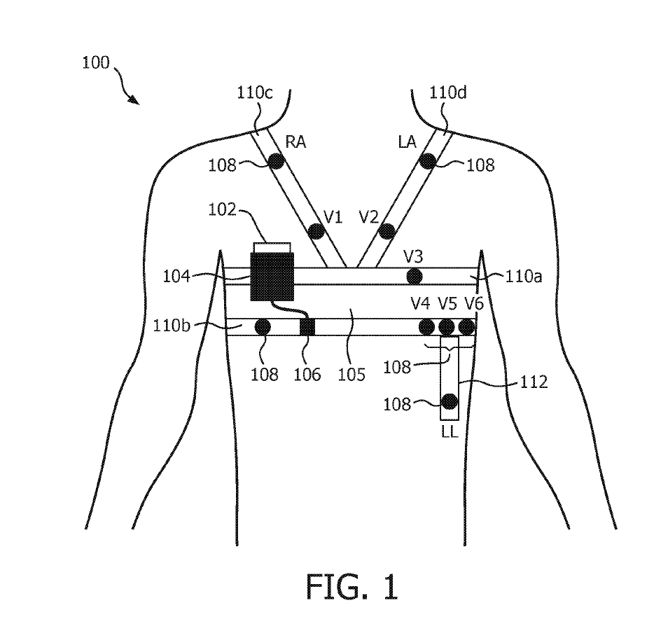

With reference to FIG. 1, a block diagram illustrating one embodiment of a patient physical monitoring system is shown. The physical monitoring system 100 includes one or more respiration rate monitoring belts 110a, 110b, supporting shoulder straps 110c, 110d assisting in supporting at least the upper monitoring belt 110a, an electronic monitoring module 102, and a module retainer 104 attached to the upper monitoring belt 110a that receives and holds the monitoring module 102. The one or more respiration belts 110a, 110b are flexible belts similar to respiration monitoring belts commonly used during sleep studies. The respiration belts can be a resistive belt that measures a patient's respiration by stretching. In the alternative, the respiration belt can also be an inductive belt that measures a patient's respiration by increasing or decreasing the area inside the belt that is wrapped around a patient. In both cases, the belts 110a, 110b are disposed around the subject's chest 105 and detect the expansion and contraction of the chest. The weight of electronic monitoring module 102 in the module retainer 104 produces force on the belt 110a; the supporting shoulder straps 110c, 110d help counter this force.

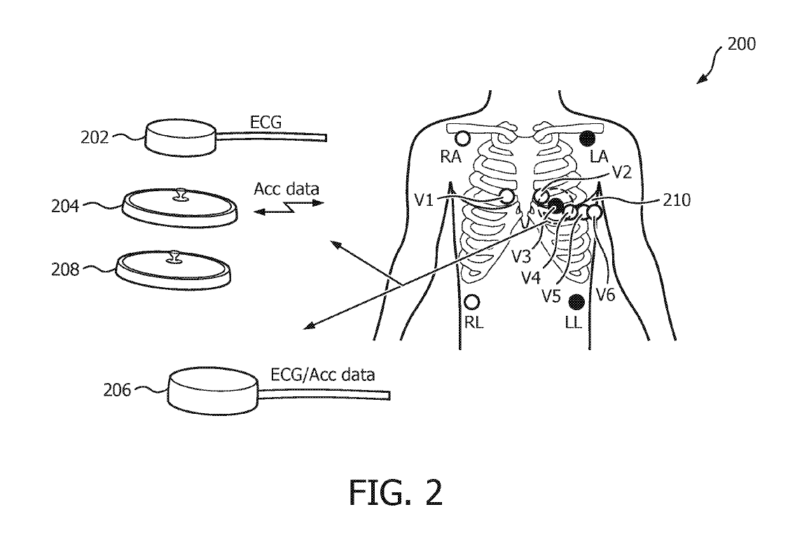

The monitoring system 100 advantageously integrates an electrocardiograph with the respiratory monitor. To this end, the one or more belts 110a, 110b and the supporting shoulder straps 110c, 110d include attached or embedded electrocardiogram (ECG) electrodes 108, with the electrode wires running through the belts 110a, 110b and shoulder straps 110c, 110d thus forming a an ECG lead wire harness that is electrically connected with the monitoring module 102. The electronic processor of the electronic monitoring device 102 is programmed to calculate the respiration rate of the patient based on the signals received from the respiration rate measurement belts 110a, 110, and is also programmed to acquire ECG traces using the ECG electrodes 108. In one embodiment, the electronic monitoring device 102 is programmed to include all or some of the following functionality. Measurement of a high resolution EGG (500 sps or better sample rate, 5 uV or better resolution), measurement of a high resolution body impedance, and input for resistive or inductive respiration belt or belts. The input can be an analog input or a radio link for a radio connected belt. In addition to the ECG electrodes 108 included in the system 100, the system can also include an accelerometer 106. The accelerometer 106 can be integrated with the ECG lead wire harness 108 so that the wired connection of the accelerometer and ECG electrode 108 is combined to form a single harness. Alternatively, the accelerometer can be built into the monitoring module 102—since the module retainer 104 holds the monitoring module 102 firmly against the torso 105, it is in proper position to acquire accelerometer data indicative of chest motion. While the accelerometer 106 is shown as a discrete element in FIG. 1 (or may be integrated with the monitoring module 102), in some embodiments the end of one, some or all ECG lead wires contain an accelerometer sitting over the ECG electrode 108 (see FIG. 2). Multiple accelerometers allow for determination of a better model for chest wall movement during respiration as well as a better—and simultaneous—model for body position such as lying down, sitting, standing, or walking.

The module retainer 104 is a pouch or other receptacle that holds the electronic monitoring module 102 firmly to the chest wall of the patient so that the electronic monitoring module 102 moves with the chest during breathing. The module retainer 104 also attaches to the one or more respiration belts and functions to hold the electronic monitoring module 102 while simultaneously measuring the chest expansion and contraction with breathing.

The illustrative physical monitoring system 100 provides a number of synergistic benefits. In the conventional 12-lead ECG electrode pattern, leads V1-V6 run approximately horizontally along the chest while the limb leads LA, RA, LL, RL are placed on the left arm, right arm, left leg, and right leg respectively. However, the limb leads in particular are very inconvenient for the patient, and accordingly modified lead placements are known, such as the Mason-Likar lead placement (see FIG. 2), which move the limb leads closer to the chest, e.g. in the Mason-Likar lead placement LA and RA are moved to the shoulders while LL and RL are moved upward onto the abdomen. As seen in FIG. 1, a close approximation to this lead layout is readily achieved in the physical monitoring system 100 by attaching or embedding the electrodes 108 for leads V1-V6 in the respiratory belts 110a, 110b, attaching or embedding the electrodes for the left and right (modified) arm leads LA, RA into the shoulder straps 110c, 110d, and providing downward extending flap or flaps 112 off the lower belt 110b to provide the left and right (modified) leg leads LL, RL. Placement of these ten electrodes 108 in their proper places is automatically achieved when the wearable frame including the one or more belts 110a, 110b and the shoulder straps 110c, 110d is placed onto the patient. If one or more accelerometers are also attached to or embedded in this wearable frame (possibly integrated with the electrodes 108 as described later with reference to FIG. 2), then these accelerometers are also precisely placed at known locations. All electrical wiring is conveniently passed through the frame elements 110a, 110b, 110c, 110d to the electronic monitoring module 102 which is held firmly to the chest wall of the patient by the module retainer 104, and support for the weight of this module 102 when the patient is ambulatory is provided by the shoulder straps 110c, 110d.

With further reference to FIG. 2, a chest diagram 200 showing the ECG electrodes V1-V6, LA, RA, LL, RL of the Mason-Likar lead placement is shown for reference. Comparison with FIG. 1 illustrates the matchup of the lead positions with the layout achievable with the frame elements 110a, 110b, 110c, 110d of the illustrative physical monitoring system 100. As further indicated in FIG. 2, an accelerometer 204 may be integrated between a disposable conductive adhesive gel ECG electrode attachment part 208 that adheres to the chest 105 and a reusable “snap-on” ECG wire terminal connector 202. The interposed accelerometer 204 may transmit accelerometer data wirelessly to the electronic monitoring module 102, or may be integrated 206 into the ECG electrode connector 202 with the ECG wire formed as a two-wire bundle: one wire carrying the ECG signal and the other the accelerometer data. In the wired embodiment the monitoring module 102 can identify the accelerometer placement directly since its signal is carried on a wire associated with the ECG electrode whose placement is known. In the wireless embodiment a suitable location header may be included in the wireless transmission.

The ECG of FIG. 1 advantageously provides 12-lead ECG traces using the Mason-Likar lead placement. Accordingly, the ECG can provide advanced electrocardiographic analyses made possible by having the complete 12-lead ECG signal set. In some embodiments the electronic monitoring module 102 is programmed to provide such analyses; at a minimum, however, the ECG provides heart rate data.

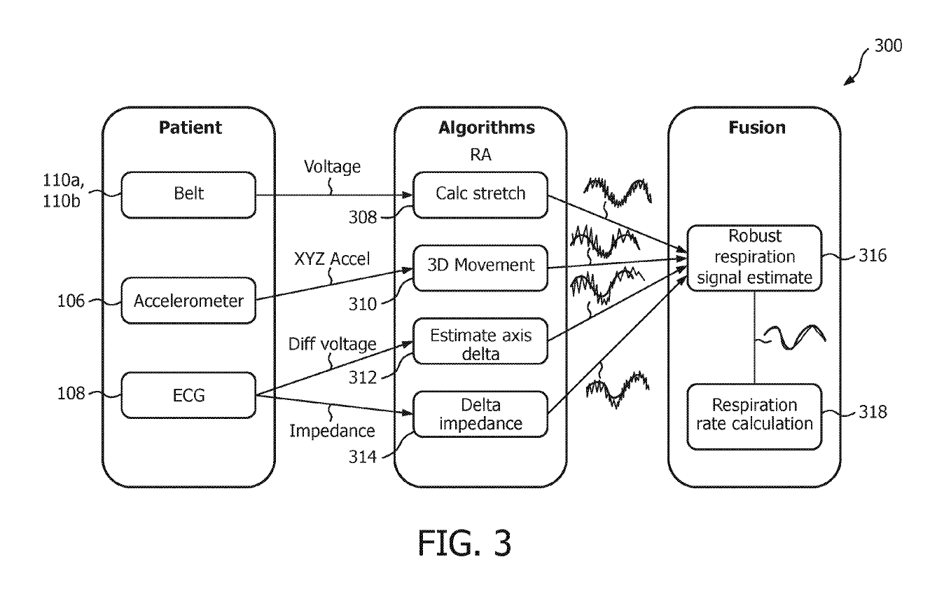

With reference back to FIG. 1 and with further reference to FIG. 3, the processor of the electronic monitoring module 102 is optionally programmed to determine a patient's respiration rate by combining a number of methods. The electronic monitoring module 102 receives measurement inputs from the respiration belt(s) 110a, 110b, the accelerometer 106, and the ECG electrodes 108, and measures and reports a patient's respiration rate based upon a combination these inputs. The electronic monitoring module 102 considers the following inputs: variation in the QRS axis from an ECG electrode input due to the diaphragm moving the heart; diaphragmatic muscle noise appearing in ECG electrodes; changing torso electrical impedance measured through a small high frequency alternating current applied to and voltage through the ECG electrodes; chest wall movement measured by accelerometer; and in resistive or inductive belt that changes due to the chest expanding and contracting due to breathing. In all cases, samples from the variation of the quantity measured constitute a digital signal which represents the cyclic inspiration and expiration of breathing. FIG. 3 shows a block diagram 300 of the inputs for respiration calculation and how the various algorithms and inputs interplay. As the belt(s) 110a, 110b stretch or inflate, the belt(s) 110a, 110b sends input information to the electronic monitoring module 102 indicating voltage change information. This information is used to determine the overall stretch of the belt either due to chest expansion or to tension 308 on the interior belt due to inflation of the belt. The electronic monitoring device 102 also receives input from the on-board accelerometer(s) 106 located in the electronic monitoring module 102 or on one of the belts 110a, 110b. The accelerometer measures the overall movement 310 of a patient's chest due to chest expansion or deflation as the patient breathes. The overall change in the position of the accelerometer is sent to the electronic monitoring module 102 as a coordinate of XYZ position change and is used to calculate the three-dimensional (3D) movement of the patient's chest. Lastly, the ECG electrodes 108 located on the one or more belts 110a, 110b and shoulder straps 110c, 110d send ECG voltage information and impedance information to the electronic monitoring module 102. This information is used to calculate the Axis Delta change 312 and the impedance change 314. While each individual respiration rate measurement method 308, 310, 312, 314 could be used in isolation to calculate a patient's respiration rate, in the approach of FIG. 3 two or more of the above described methods are combined to determine the respiration rate. Any combination of two, three, or all four of the methods 308, 310, 312, 314 may be used to produce a robust respiration signal estimate 316 by fusing the measurements. The electronic monitoring device 100 calculates a patient's respiration rate in fusion operation 318 using all available respiration parameters including the complete set or a subset of ECG derived respiration, impedance based respiration, accelerometer based respiration and chest belt respiration. One potential way to combine the respiration signals into a single representative signal is by periodic principal component analysis. The largest component would be an estimate of the true signal while the other components would be noise components.

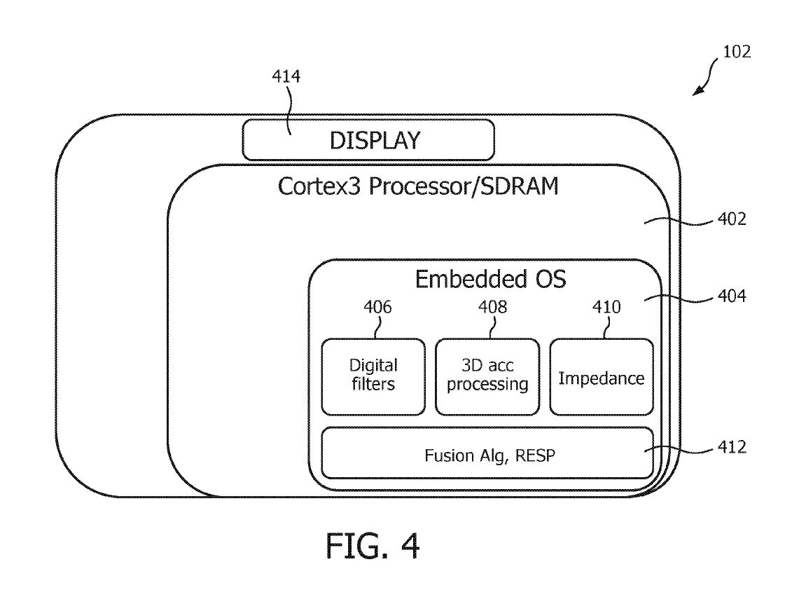

With reference to FIG. 4, a suitable architecture of the electronic monitoring module 102 is shown. The monitoring module 102 includes memory 402 with an embedded operating system 404. The embedded operating system 404 receives the patient measurements from the belts, the ECG electrodes, and the accelerometer. The various inputs 406, 408, 410 are stored and used by the operating system 404. The resulting patient respiration is calculated using at least two or more of the above inputs 406, 408, 410. The Fusion Alg, RESP module 412 retrieves the inputs from memory and calculates the resulting respiration.

The pouch or other module retainer 104 can be variously configured. In one approach, the module retainer 104 includes a conformal sleeve into which the monitoring device slides, and an electrical connector at the bottom of the sleeve into which a mating electrical connector of the monitoring module 102 engages to make simultaneous electrical connection with the ECG, respiratory belts, and accelerometers (if they have a wired connection). The electronic monitoring module 102 preferably further includes a display 414 via which the calculated respiration rate is displayed to a user.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A physical monitoring system comprising:
one or more resistive or inductive respiration belts configured to be disposed around a chest to detect chest expansion and contraction during breathing;
an electronic monitoring module operatively connected with the one or more resistive or inductive respiration belts and comprising a processor programmed to compute respiration using the one or more resistive or inductive respiration belts;
one or more accelerometers attached to or embedded in the one or more resistive or inductive respiration belts and connected with the electronic monitoring module via wires passing through the belts; and
a module retainer receiving the electronic monitoring module and securing the electronic monitoring module to the one or more resistive or inductive respiration belts;
wherein the processor is configured to determine a weighted average of a plurality of signals which represent respiratory chest expansion and contraction generated from the belts and the at least one of the ECG and the accelerometer, and calculate a respiration rate from a fusion signal generated by principal component analysis of the plurality of signals for the weighted average.

2. The physical monitoring system of claim 1 further comprising:
electrocardiogram (ECG) electrodes attached to or embedded in the one or more resistive or inductive respiration belts whereby the ECG electrodes assume a desired ECG electrodes layout on a subject when the one or more resistive or inductive respiration belts are disposed around the subject, the ECG electrodes connected with the electronic monitoring module via wires passing through the belts.

3. The physical monitoring system of claim 2 further comprising:
shoulder straps supporting at least one of the one or more resistive or inductive respiration belts; and
modified left and right arm ECG electrodes attached to or embedded in the shoulder straps.

4. The physical monitoring system of claim 3 further comprising:
downward extending straps extending downward from the one or more resistive or inductive respiration belts; and
modified left and right leg ECG electrodes attached to or embedded in the downward extending straps;
wherein the ECG electrodes attached to or embedded in the belts comprise ECG electrodes V1-V6 such that the ECG electrodes attached to or embedded in the belts, shoulder straps, and downward extending straps form a Mason-Likar lead placement.

5. (canceled)

6. (canceled)

7. (canceled)

8. The physical monitoring system according to claim 1, wherein the module retainer comprises a flexible pouch.

9. (canceled)

10. The physical monitoring system according to claim 1, wherein the respiratory rate measurements include at least one of: variation in the QRS axis in the ECG due to movement in the heart; diaphragmatic muscle noise on the ECG; a change in torso electrical impedance measured through ECG electrodes; chest wall movement measured by the accelerometer; and resistive or inductive belt changes due to chest expansion.

11. The system according to claim 1, wherein the electronic monitoring module includes:
a display via which the calculated respiration rate is displayed to a user.

12. A physical monitoring system comprising:
one or more resistive or inductive respiration belts;
electrocardiogram (ECG) electrodes attached to or embedded in the one or more resistive or inductive respiration belts;
an electronic monitoring module with an on-board accelerometer attached to the one or more resistive or inductive respiration belts and to the ECG electrodes via wires passing through the one or more resistive or inductive respiration belts, the electronic monitoring module programmed to compute respiration using at least the one or more resistive or inductive respiration belts and to compute at least heart rate using the ECG electrodes; and
a module retainer configured to receive the electronic monitoring module and to secure the electronic monitoring module to the one or more resistive or inductive respiration belts.

13. The physical monitoring system of claim 12, wherein the electronic monitoring module is configured to compute a respiration rate based on the accelerometer signal.

14. The physical monitoring system according claim 12 wherein an ECG lead includes a disposable conductive adhesive gel ECG electrode attachment part and a reusable ECG wire terminal connector, and the physical monitoring system further comprises:
an accelerometer disposed between the disposable conductive adhesive gel ECG electrode attachment part and the reusable ECG wire terminal connector.

15. The physical monitoring system according to claim 13, wherein the electronic monitoring module is configured to:
determine a weighted average of all respiration signal estimates derived from the ECG electrodes and accelerometer;
calculate an average for all received weighted averages;
use principal component analysis to create weights for the weighted averages; and
calculate respiration rate from a cyclic respiratory signal generated by the weighted averages.

16. The physical monitoring system according to claim 12 further comprising:
shoulder straps supporting at least one of the one or more resistive or inductive respiration belts; and
ECG electrodes attached to or embedded in the shoulder straps;
wherein the ECG electrodes attached to or embedded in the one or more resistive or inductive respiration belts and the ECG electrodes attached to or embedded in the shoulder straps together define a modified 12-lead ECG lead placement.

17. The physical monitoring system according to claim 16 wherein the one or more resistive or inductive respiration belts include one or more downward extending flaps with ECG electrodes attached to or embedded in the downward extending flaps to define LL and RL electrodes of the 12-lead ECG lead placement.

18. (canceled)

19. (canceled)

20. (canceled)
